(19) United States
(12) Patent Application Publication
(10) Pub. No.: US 2014/0199299 A1
(43) Pub. Date: Jul. 17, 2014

(54) Combination Therapies and Methods Using Anti-CD3 Modulating Agents and Anti-IL-6 Antagonists

(71) Applicant: Novlmmune S.A., Geneva (CH)

(72) Inventors: Yann Dean, Bossey (FR); Fabien Depis, Paris (FR)

(21) Appl. No.: 14/220,863

(22) Filed: Mar. 20, 2014

Related U.S. Application Data
(63) Continuation of application No. 13/354,813, filed on Jan. 20, 2012, now abandoned.
(60) Provisional application No. 61/434,874, filed on Jan. 21, 2011.

(51) Int. Cl. A61K 39/395

(52) U.S. Cl. CPC A61K 39/39558; A61K 39/3955
USPC 424/133.1; 424/142.1; 424/145.1; 424/158.1

(57) ABSTRACT

This invention relates generally to compositions that contain multiple modulating agents, e.g., multiple modulating agents that target CD3 on T cells and neutralize one or more biological activities of interleukin-6 (IL-6), such as CD3 modulators including anti-CD3 antibodies and anti-IL-6 antagonists including anti-IL-6 antibodies, anti-IL-6R antagonists including anti-IL-6R antibodies, and/or anti-IL-6/IL-6R complex antagonists including anti-IL-6/IL-6R binding antibodies, and methods of using these compositions in the treatment, amelioration and/or prevention of relapse of an autoimmune disease.

### RELATED APPLICATIONS

This application is a continuation of U.S. patent application Ser. No. 13/354,813, filed Jan. 20, 2012, which claims the benefit of and priority to U.S. Provisional Patent Application No. 61/434,874, filed Jan. 21, 2011, which is herein incorporated by reference in its entirety.

### FIELD OF THE INVENTION

This invention relates generally to compositions that contain multiple pharmaceutical reagents, such as modulating agents, e.g., multiple neutralizing monoclonal antibodies, that target CD3 on T cells and neutralize one or more biological activities of interleukin-6 (IL-6), such as CD3 modulators and anti-IL-6 antagonists, and methods of using these compositions in the treatment, amelioration and/or prevention of relapse of an autoimmune disease.

### INCORPORATION-BY-REFERENCE OF SEQUENCE LISTING

The contents of the text file named “425001USSeqList.txt,” which was created on Jan. 11, 2011 and is 7.63 KB in size, are hereby incorporated by reference in their entirety.

### BACKGROUND OF THE INVENTION

Interleukin 6 (IL-6) is a potent pleiotropic cytokine that regulates cell growth and differentiation and is also an important mediator of acute inflammatory responses. IL-6 exhibits its action via a receptor complex consisting of a specific IL-6 receptor (IL-6R) and a signal transducing subunit (gp130). Dysregulated IL-6 signaling has been implicated in the pathogenesis of many diseases, such as multiple myeloma, autoimmune diseases and prostate cancer. Accordingly, there exists a need for therapies that neutralize the biological activities of IL-6 and/or IL-6R.

### SUMMARY OF THE INVENTION

The present invention provides combination therapies and methods that use modulators of CD3 and antagonists of interleukin 6 (IL-6), interleukin 6 receptor (IL-6R) and/or the complex formed by IL-6 and IL-6R (also referred to herein as the IL-6/IL-6R complex) to treat, delay the progression of, prevent relapse of or alleviate a symptom of an autoimmune disease. The combination therapies are capable of modulating, e.g., blocking, inhibiting, reducing, antagonizing, neutralizing or otherwise interfering with one or more biological activities of IL-6, IL-6R and/or the IL-6/IL-6R complex, also referred to herein as “IL-6Rc.” The combination therapies are also capable of modulating or otherwise altering at least one biological function of CD3 and/or the complex formed between CD3 and T cell receptor (also referred to herein as the CD3/TcR complex).

The combination therapies of the invention are capable of modulating, e.g., blocking, inhibiting, reducing, antagonizing, neutralizing or otherwise interfering with IL-6R intracellular signaling via activation of the JAK/STAT pathway and MAPK cascade. Binding of IL-6 and IL-6R to form the IL-6/IL-6R complex (IL-6Rc). The IL-6Rc interacts or otherwise associates with gp130, a transmembrane glycoprotein. In particular, binding of IL-6 to IL-6R leads to disulfide-linked homodimerization of gp130 within a cell, which, in turn, leads to the activation of a tyrosine kinase as the first step in signal transduction. In a preferred embodiment, the combination therapies of the invention bind to IL-6Rc and block or otherwise inhibit IL-6Rc from interacting with gp130, thereby preventing, partially or completely, the homodimerization of gp130 and subsequent signaling (cis and trans).

IL-6 acts as both a pro-inflammatory and anti-inflammatory cytokine. It is secreted by T cells and macrophages to stimulate immune response to trauma, especially burns or other tissue damage leading to inflammation. IL-6 is one of the most important mediators of fever and of the acute phase response. In the muscle and fatty tissue IL-6 stimulates energy mobilization which leads to increased body temperature. IL-6 can be secreted by macrophages in response to specific microbial molecules, referred to as pathogen associated molecular patterns (PAMPs). These PAMPs bind to highly important detection molecules of the innate immune system, called Toll-like receptors (TLRs), that are present on the cell surface (or in intracellular compartments) which induce intracellular signaling cascades that give rise to inflammatory cytokine production. IL-6 is also essential for hybridoma growth and is found in many supplemental cloning media such as briclone.

IL-6 signals through a cell-surface type I cytokine receptor complex consisting of the ligand-binding IL-6Rα chain (also known as CD126), and the signal-transducing component gp130 (also called CD130). gp130 is the common signal transducer for several cytokines including leukemia inhibitory factor (LIF), ciliary neurotrophic factor, oncostatin M, IL-11 and cardiotrophin-1, and is almost ubiquitously expressed in most tissues. In contrast, the expression of CD126 is restricted to certain tissues. As IL-6 interacts with its receptor, it triggers the gp130 and IL-6R proteins to form a complex, thus activating the receptor. These complexes bring together the intracellular regions of gp130 to initiate a signal transduction cascade through certain transcription factors, Janus kinases (JAKs) and Signal Transducers and Activators of Transcription (STATs). Accordingly, neutralization of IL-6 signaling is a potential therapeutic strategy in the treatment of disorders such as, for example, sepsis, cancer (e.g., multiple myeloma disease (MM), renal cell carcinoma (RCC), plasma cell leukaemia, lymphoma, B-lymphoproliferative disorder (BLPD), and prostate cancer), bone resorption, osteoporosis, cachexia, psoriasis, mesangial proliferative glomerulonephritis, Kaposi's sarcoma, AIDS-related lymphoma, and inflammatory diseases (e.g., rheumatoid arthritis (RA), systemic onset juvenile idiopathic arthritis, hypergammaglobulinemia, Crohn's disease (CD), ulcerative colitis, systemic lupus erythematosus (SLE), multiple sclerosis, Castleman's disease, IgM gammopathy, cardiac myxoma, asthma, allergic asthma and autoimmune insulin-dependent diabetes mellitus).

A clinically proven way to alter T cell function is by the administration of anti-CD3 antibodies. The mechanism involves modulation of the CD3/TcR complex from the T cell surface and a transient disappearance of lymphocytes from the circulation. Anti-CD3 treatment in NOD mice, a model of Type 1 diabetes, has been shown to not only eliminate pathogenic effector T cells but also induce concomitantly TGF-β dependent T regulatory cells. (Chatenoud L, Thervet E, Primo J, Bach J F. Anti-CD3 antibody induces long-term remission of overt autoimmunity in nonobese diabetic mice. (1994) PNAS. 91: 123-127; You. S et al. Adaptive TGF-β-dependent regulatory T cells control autoimmune diabetes and are a privileged target of anti-CD3 antibody treatment. (2007) PNAS. 104: 6335-6340; and Perruche S, Bluestone J. A, Wanjun C et al. CD3-specific antibody-induced immune tolerance involves transforming growth factor-β from phagocytes digesting apoptotic T cells. (2008) Nature. Med. 14: 528-535). However, the efficacy of anti-CD3 treatment in arthritis remains controversial (Maeda T et al. Exacerbation of established collagen-induced arthritis in mice treated with an anti-T cell receptor antibody. (1994) Arthritis. Rheum. 37: 406-413; Hughes et al. Induction of T helper cell hyporesponsiveness in an experimental model of autoimmunity by using nonmitogenic anti-CD3 monoclonal antibody. (1994) J. Immunol. 153: 3319-3325; Pietersz G A et al. Inhibition of destructive autoimmune arthritis in FcgammaRIIa transgenic mice by small chemical entities. (2009) Immunol Cell Biol. 87:3-12; and Malfait A M et al. Chronic relapsing homologous collagen-induced arthritis in DBA/1 mice as a model for testing disease-modifying and remission-inducing therapies. (2001) Arthritis. Rheum. 44: 1215-1224), once again underlining the multi-factorial nature of autoimmune diseases.

Combination therapy with two modulating agents, for example, two monoclonal antibodies (mAbs), one that targets CD3 on T cells and one that neutralizes IL-6, IL-6R and/or the complex formed between IL-6 and IL/6R referred to herein as IL-6Rc, produces a potent synergy that reduces disease severity and prevents disease relapse, as shown in FIG. 1. Combination therapies are not limited to monoclonal antibodies and can include any agent that modulates CD3 and any antagonist of IL-6, IL-6R and/or the IL-6Rc. This finding provides the basis to support using such a combination strategy to obtain an effective long-term treatment for RA, CD and other autoimmune diseases.

The combination therapies provided herein are useful for treating, delaying the progression of, preventing a relapse of, or alleviating a symptom of an autoimmune disease by administering a combination of reagents such as modulating agents to a subject in need thereof in an amount sufficient to treat, delay the progression of, prevent a relapse of, or alleviate the symptom of the autoimmune disease in the subject, wherein the combination of modulating agents comprises a modulating agent that binds to or otherwise interacts with CD3 and an antagonist that binds to or otherwise interacts with IL-6, IL-6R and/or the IL-6Rc. As used herein, the term “modulating agent” refers to a reagent that binds to or otherwise interacts with a target, e.g., CD3, and alters at least one biological property and/or biological activity of that target. The terms “modulating agent” and “modulator” are used interchangeably herein. The CD3 modulators, also referred to herein as CD3 modulating agents and modulators of CD3, bind CD3 and alter or otherwise modify at least one biological property and/or biological activity of that target. In some embodiments, the CD3 modulators used in the combination therapies provided herein have an inhibitory or otherwise neutralizing effect on at least one biological property and/or biological activity of CD3 and also have a stimulatory effect on at least a second biological property and/or biological activity of CD3. For example, in some embodiments, the CD3 modulator binds or otherwise interacts with CD3 and alters (e.g., decreases) the cell surface expression level or activity of CD3 or the T cell receptor (TcR). In some embodiments, exposure to the CD3 modulating agent removes or masks CD3 and/or TcR without affecting cell surface expression of CD2, CD4 or CD8. The masking of CD3 and/or TcR results in the loss or reduction of T-cell activation, which is desirable in autoimmune diseases where uncontrolled T-cell activation occurs. Antigenic modulation refers to the redistribution and elimination of the CD3-T cell receptor complex on the surface of a cell, e.g., a lymphocyte. Decrease in the level of cell surface expression or activity of the TcR on the cell is meant that the amount or function of the TcR is reduced. Modulation of the level of cell surface expression or activity of CD3 is meant that the amount of CD3 on the cell surface or function of CD3 is altered, e.g., reduced. The amount of CD3 or the TcR expressed at the plasma membrane of the cell is reduced, for example, by internalization of CD3 or the TcR upon contact of the cell with the CD3 modulator. Alternatively, upon contact of a cell with the CD3 modulating agent, CD3 or the TcR is masked.

In some embodiments, the modulator of CD3 is an anti-CD3 antibody. In some embodiments, the anti-CD3 antibody is a monoclonal antibody (mAb). In some embodiments, the anti-CD3 antibody is a mouse, chimeric, humanized, fully human mAb, domain antibody, single chain, F_{ab}, F_{ab}, and F_{(ab′)2} fragments, scFvs, or an F_{ab} expression library.

In some embodiments, the anti-CD3 antibody is the fully human anti-CD3 mAb referred to herein as “NI-0401,” “Foralumab” and/or “28F11,” which includes a heavy chain CDR1 having the amino acid sequence GYGMH (SEQ ID NO: 1), a heavy chain CDR2 having the amino acid sequence VIWYDGSKKYYVDSVKG (SEQ ID NO: 2), a heavy chain CDR3 having the amino acid sequence QMGYWHFDL (SEQ ID NO: 3), a light chain CDR1 having the amino acid sequence RASQSVSSYLA (SEQ ID NO: 4), a light chain CDR2 having the amino acid sequence DASNRAT (SEQ ID NO: 5), and a light chain CDR3 having the amino acid sequence QQRSNWPPLT (SEQ ID NO: 6). In some embodiments, the NI-0401 antibody comprises a variable heavy chain region comprising the amino acid sequence shown below and a variable light chain region comprising the amino acid sequence shown below.

>NI-0401 VH nucleotide sequence:
(SEQ ID NO: 7)
CAGGTGCAGCTGGTGGAGTCCGGGGGAGGCGTGGTCCAGCCTGGGAGGTCCCTGAGACTCTCCT
GTGCAGCGTCTGGATTCAAGTTCAGTGGCTATGGCATGCACTGGGTCCGCCAGGCTCCAGGCAA
GGGGCTGGAGTGGGTGGCAGTTATATGGTATGATGGAAGTAAGAAATACTATGTAGACTCCGTG
AAGGGCCGCTTCACCATCTCCAGAGACAATTCCAAGAACACGCTGTATCTGCAAATGAACAGCC
TGAGAGCCGAGGACACGGCTGTGTATTACTGTGCGAGACAAATGGGCTACTGGCACTTCGATCT
CTGGGGCCGTGGCACCCTGGTCACTGTCTCCTCA
>NI-0401 VH amino acid sequence:
(SEQ ID NO: 8)

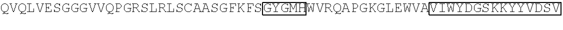

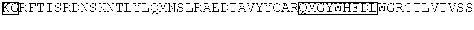

>NI-0401 VL nucleotide sequence:
(SEQ ID NO: 9)
GAAATTGTGTTGACACAGTCTCCAGCCACCCTGTCTTTGTCTCCAGGGGAAAGAGCCACCCTCT
CCTGCAGGGCCAGTCAGAGTGTTAGCAGCTACTTAGCCTGGTACCAACAGAAACCTGGCCAGGC
TCCCAGGCTCCTCATCTATGATGCATCCAACAGGGCCACTGGCATCCCAGCCAGGTTCAGTGGC
AGTGGGTCTGGGACAGACTTCACTCTCACCATCAGCAGCCTAGAGCCTGAAGATTTTGCAGTTT
ATTACTGTCAGCAGCGTAGCAACTGGCCTCCGCTCACTTTCGGCGGAGGGACCAAGGTGGAGAT
CAAA
>NI-0401 VL amino acid sequence:
(SEQ ID NO: 10)

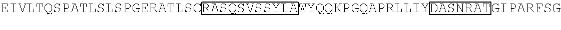

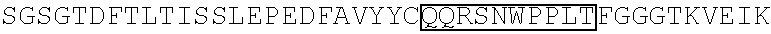

In some embodiments, the NI-0401 antibody further includes a mutation in the heavy chain at an amino acid residue at position 234, 235, 265, or 297 or combinations thereof, and reduces the release of cytokines from a T-cell. In some embodiments, the mutation results in an alanine or glutamic acid residue at the position. In some embodiments, the NI-0401 antibody is an IgG1 isotype and contains at least a first mutation at position 234 and a second mutation at position 235, wherein the first mutation results in an alanine residue at position 234 and the second mutation results in a glutamic acid residue at position 235.

In some embodiments, the anti-CD3 modulating agent is a fully human anti-CD3 mAb. Suitable antibodies for use in the combination therapies and methods provided herein include, by way of non-limiting example, those antibodies described in PCT Publication No. WO 05/118635, the contents of which are hereby incorporated by reference in their entirety, or an anti-CD3 antibody that binds to the same epitope as those antibodies described in PCT Publication No. WO 05/118635. Other suitable anti-CD3 mAbs for use in the combination therapies and methods provided herein include, but are not limited to, Orthoclone OKT3 (also known as Muromonab), human OKT3γ1 (HOKT3γ1, also known as Teplizumab), ChAglyCD3 (also known as Otelixizumab) and Nuvion® (also known as Visilizumab), or antibodies that bind to the same epitope as Orthoclone OKT3, human OKT3γ1 (HOKT3γ1), ChAglyCD3 or Nuvion® (Visilizumab).

In some embodiments, the anti-CD3 antibody contains an amino acid mutation. For example, the mutation is in the constant region. Preferably, the mutation results in an antibody that has an altered effector function. An effector function of an antibody is altered by altering, i.e., enhancing or reducing, the affinity of the antibody for an effector molecule such as an Fc receptor or a complement component. By altering an effector function of an antibody, it is possible to control various aspects of the immune response, e.g., enhancing or suppressing various reactions of the immune system. For example, the mutation results in an antibody that is capable of reducing cytokine release from a T-cell. For example, the mutation is in the heavy chain at amino acid residue 234, 235, 265, or 297 or combinations thereof. Preferably, the mutation results in an alanine residue at either position 234, 235, 265 or 297, or a glutamate residue at position 235, or a combination thereof. The term “cytokine” refers to all human cytokines known within the art that bind extracellular receptors expressed on the cell surface and thereby modulate cell function, including but not limited to IL-2, IFN-gamma, TNF-a, IL-4, IL-5, IL-6, IL-9, IL-10, and IL-13.

The release of cytokines can lead to a toxic condition known as cytokine release syndrome (CRS), a common clinical complication that occurs, e.g., with the use of an anti-T cell antibody such as ATG (anti-thymocyte globulin) and OKT3 (a murine anti-human CD3 antibody). This syndrome is characterized by the excessive release of cytokines such as TNF, IFN-gamma, IL-6 and IL-2 into the circulation. The CRS occurs as a result of the simultaneous binding of the antibodies to CD3 (via the variable region of the antibody) and the Fc Receptors and/or complement receptors (via the constant region of the antibody) on other cells, thereby activating the T cells to release cytokines that produce a systemic inflammatory response characterized by hypotension, pyrexia and rigors. Symptoms of the CRS include fever, chills, nausea, vomiting, hypotension, and dyspnea. Thus, the anti-CD3 antibody used in some embodiments of the combination therapy contains one or more mutations that prevent heavy chain constant region-mediated release of one or more cytokine(s) in vivo.

The anti-CD3 antibodies used in the combination therapies provided herein include, for example, a L²³⁴L²³⁵→A²³⁴E²³⁵ mutation in the Fc region, such that cytokine release upon exposure to the anti-CD3 antibody is significantly reduced or eliminated, as described in PCT Publication No. WO 05/118635. Other mutations in the Fc region include, for example, L²³⁴L²³⁵→A²³⁴A²³⁵, L²³⁵→E²³⁵, N²⁹⁷→A²⁹⁷, and D²⁶⁵→A²⁶⁵.

In some embodiments, the modulator of CD3 is a modified antibody reagent or a non-antibody-based reagent. Such modulators include advanced antibody therapeutics, such as bispecific antibodies, immunotoxins, and radiolabeled therapeutics; peptide therapeutics; gene therapies, particularly intrabodies; oligonucleotide therapeutics such as aptamer therapeutics, antisense therapeutics, interfering RNA therapeutics; and small molecules.

The combination therapies provided herein include an antagonist of IL-6, an antagonist of IL-6R, an antagonist of IL-6Rc or combinations thereof. In some embodiments, the combination therapy includes an antagonist of IL-6. In some embodiments, the antagonist of IL-6 is an anti-IL-6 antibody. In some embodiments, the anti-IL-6 antibody is a mAb. For example, the anti-IL-6 antibody is a chimeric, humanized, domain and/or fully human mAb. The antibodies bind to an IL-6 and/or IL-6Rc epitope with an equilibrium binding constant (K_{d}) of ≦1 μM, e.g., ≦100 nM, preferably ≦10 nM, and more preferably ≦1 nM. For example, the anti-IL-6 antibodies used in the combination therapies provided herein exhibit a K_{d} in the range approximately between ≦1 nM to about 1 pM.

In some embodiments, the combination therapy includes an antagonist of IL-6R. In some embodiments, the antagonist of IL-6R is an anti-IL-6R antibody. In some embodiments, the anti-IL-6R antibody is a mAb. For example, the anti-IL-6R antibody is a chimeric, humanized, domain and/or fully human mAb. The antibodies bind to an IL-6R and/or IL-6Rc epitope with an equilibrium binding constant (K_{d}) of ≦1 μM, e.g., ≦100 nM, preferably ≦10 nM, and more preferably ≦1 nM. For example, the anti-IL-6R antibodies used in the combination therapies exhibit a K_{d} in the range of approximately between ≦1 nM to about 1 pM.

In some embodiments, the combination therapy includes an antagonist of IL-6. In some embodiments, the antagonist of IL-6Rc is an anti-IL-6 antibody. In some embodiments, the anti-IL-6Rc antibody is a mAb. For example, the anti-IL-6Rc antibody is a chimeric, humanized, domain and/or fully human mAb. The antibodies bind to an IL-6, IL-6R and/or IL-6Rc epitope with an equilibrium binding constant (K_{d}) of ≦1 μM, e.g., ≦100 nM, preferably ≦10 nM, and more preferably ≦1 nM. For example, the anti-IL-6Rc antibodies used in the combination therapies provided herein exhibit a K_{d} in the range approximately between ≦1 nM to about 1 pM.

Antibodies for use in the combination therapies provided herein include antibodies that bind the human IL-6/IL-6R complex and also bind IL-6 independently of the presence of IL-6R. Antibodies for use in the combination therapies provided herein also include antibodies that bind the human IL-6/IL-6R complex and also bind IL-6R independently of the presence of IL-6. Antibodies for use in the combination therapies provided herein also include antibodies that bind the IL-6 portion of the human IL-6/IL-6R complex, but binding is entirely dependent on the presence of IL-6R. Antibodies for use in the combination therapies provided herein also include antibodies that bind the IL-6R portion of the human IL-6/IL-6R complex, but binding is entirely dependent on the presence of IL-6.

In some embodiments, the combination therapy includes one or more anti-IL-6 antibodies that recognize membrane bound human IL-6 when complexed with the human IL-6 receptor (IL-6R), which is also known as the human IL-6/IL-6R complex (“IL-6Rc”). These anti-IL-6 antibodies recognize IL-6Rc expressed on the cell surface and/or in soluble form. The anti-IL-6 antibodies are capable of modulating, e.g., blocking, inhibiting, reducing, antagonizing, neutralizing or otherwise interfering with IL-6R intracellular signaling via activation of the JAK/STAT pathway and MAPK cascade. Anti-IL-6 antibodies useful in the combinations provided herein also include antibodies that bind soluble IL-6Rc. In addition, combination therapies of the invention include antibodies that bind IL-6Rc, wherein they also bind human IL-6R alone (i.e., when not complexed with IL-6).

In some embodiments, the anti-IL-6, anti-IL-6R and/or anti-IL-6Rc antibodies used in the combinations described herein bind the complex formed by IL-6R and IL-6 and thereby prevent the binding of the IL-6/IL-6R complex (“IL-6Rc”) to the transmembrane glycoprotein gp130 and subsequent signaling (both cis and trans), which is activated by the IL-6Rc/gp130 signaling complex.

In some embodiments, the antibodies used in the combinations provided herein modulate, e.g., block, inhibit, reduce, antagonize, neutralize or otherwise interfere with, the interaction between the IL-6Rc and gp130.

In some embodiment, the antibodies used in the combinations described herein bind to IL-6 or IL-6R individually, for example, in the groove where IL-6 binds to IL-6R and inhibit or otherwise interfere with the interaction between IL-6 and IL-6R and prevent the formation of IL-6Rc.

In some embodiments, the anti-IL-6, anti-IL-6R, and/or anti-IL-6Rc antibody or immunologically active fragment thereof is, or is derived from, an antibody as described in PCT/US2009/043734, filed May 13, 2009 and published as WO 2009/140348, the contents of which are hereby incorporated by reference in their entirety.

In some embodiments, the combination therapy includes an anti-IL-6 antagonist. For example, in some embodiments, the anti-IL-6 antagonist is an antibody, such as a commercially available antibody, including, for example, CNTO 328 (an anti-IL-6 chimeric monoclonal antibody, see e.g., Pinski et al., J. Clin. Oncol., vol. 27 (Suppl. 15): A-5143 (2009)), also known as siltuximab (Centocor, Inc., Johnson & Johnson, see e.g., U.S. Pat. No. 7,291,721); CDP6038 (UCB S.A.), MEDI5117, an affinity-optimized human anti-IL-6 monoclonal antibody IgG1 which incorporates YTE Fc modification to extend its plasma half-life (MedImmune, AstraZeneca, see e.g., Moisan, et al, “MEDI5117: A Human High Affinity Anti-IL-6 Monoclonal Antibody with Enhanced Serum Half-Life in Development for the Treatment of Inflammation and Rheumatological Diseases [abstract],” Arthritis Rheum; vol. 60 (Suppl 10): 401 (2009)), ALD518 (also known as BMS-945429) which is an aglycosylated, humanized monoclonal IgG1 antibody against interleukin-6 (Bristol Myers Squibb, also known as BMS-945429), FM101, a femto molar binding antibody that is directed against IL-6 (Femta Pharmaceuticals, Lonza) or Elsilimomab (also known as B-E8, an murine anti-human IL6 mAb or its fully human counterpart mAb 1339 (also known as OP-R003 or Azintrel, see e.g., Fulciniti et al., “A high-affinity fully human anti-IL-6 mAb, 1339, for the treatment of multiple myeloma,” Clin Cancer Res., vol. 15(23):7144-52 (2009).

In some embodiments, the anti-IL-6 antagonist is an antagonist peptide, polypeptide or protein such as, for example, C326 (an IL-6 inhibitor by Avidia, also known as AMG220), or FE301, a recombinant protein inhibitor of IL-6 (Ferring International Center S.A., Conaris Research Institute AG).

In some embodiments, the anti-IL-6 antagonist is soluble gp130.

In some embodiments, the combination therapy includes an anti-IL-6R antagonist. For example, in some embodiments, the anti-IL-6R antagonist is an antibody or is derived from an antibody. For example, in some embodiments, the antil-IL-6R antagonist is a domain antibody such as for example, the Nanobody™ ALX0061 (Ablynx). In some embodiments, the anti-IL-6R antagonist is an antibody, for example a humanized antibody such as Tocilizumab, also known as actemra, which is a humanized anti-IL-6R mAb that blocks IL-6 signaling (Chugai, Roche, see e.g., Drug Ther Bull., “Tocilizumab for rheumatoid arthritis,” vol. 48(1):9-12 (2010)). In some embodiments, the anti-IL-6R antagonist is an antibody, for example a human mAb such as REGN88 (Regeneron).

In some embodiments, the anti-IL-6R antagonist is a peptide, polypeptide or protein-based antagonist. For example, the anti-IL-6R antagonist is X1 TNFR X ANTI-IL-6/IL-6R—SCORPION™ therapeutic, a single chain protein (Emergent BioSolutions Inc., see e.g., Next Generation Protein Therapeutics Conference September 2010; “SMIP and SCORPION Proteins: Novel, Mono or Multi-Specific Therapeutic Proteins for Autoimmune Diseases and Oncology;” Kendall M. Mohler, Ph.D.)

Exemplary monoclonal antibodies for use in the combination therapies of the invention include monoclonal antibodies that bind to human IL-6, human IL-6R and/or human IL-6Rc. These antibodies are respectively referred to herein as “huIL-6Rc” antibodies. hulL-6Rc antibodies include fully human monoclonal antibodies, as well as humanized monoclonal antibodies, domain antibodies, and chimeric antibodies. In some embodiments, the antibodies show specificity for human IL-6Rc and IL-6R. In some embodiments, the antibodies modulate, e.g., block, inhibit, reduce, antagonize, neutralize or otherwise interfere with IL-6Rc mediated intracellular signaling (cis and/or trans signaling).

In some embodiments, the anti-IL-6, IL-6R and/or IL-6Rc antibody includes the amino acid sequence RASQGISSVLA (SEQ ID NO: 12) in the light chain CDR1 region, the amino acid sequence DASSLES (SEQ ID NO: 13) in the light chain CDR2 region, the amino acid sequence QQSNSYPLT (SEQ ID NO: 14) in the light chain CDR3 region, the amino acid sequence SYAIS (SEQ ID NO: 15) in the heavy chain CDR1 region, the amino acid sequence GIIPLFDTTKYAQKFQG (SEQ ID NO: 16) in the heavy chain CDR2 region, the amino acid sequence DRDILTDYYPMGGMDV (SEQ ID NO: 17) in the heavy chain CDR3 region, and the amino acid sequence TAVYYCAR (SEQ ID NO: 18) in the FRW3 region. In some embodiments, the anti-IL-6, IL-6R and/or IL-6Rc antibody includes a variable heavy chain region that includes the amino acid sequence QVQLVQSGAEVKKPGSSVKVSCKASGGTFSSYAISWVRQAPGQGLEWMGGIIPLFDTTK YAQKFQGRVTITADESTSTAYMELSSLRSEDTAVYYCARDRDILTDYYPMGGMDVWG QGTTVTVSS (SEQ ID NO: 19), and a variable light chain region that includes the amino acid sequence AIQLTQSPSSLSASVGDRVTITCRASQGISSVLAWYQQKPGKAPKLLIYDASS LESGVPSRFSGSGSGTDFTLTISSLQPEDFATYYCQQSNSYPLTFGGGTKVEIKR (SEQ ID NO: 20). This antibody is referred to herein as the NI-1201A antibody.

The three heavy chain CDRs include a variable heavy chain (VH) complementarity determining region 1 (CDR1) that includes an amino acid sequence at least 90%, 92%, 95%, 97% 98%, 99% or more identical to a sequence shown herein; a VH complementarity determining region 2 (CDR2) that includes an amino acid sequence at least 90%, 92%, 95%, 97% 98%, 99% or more identical to a sequence shown herein; and a VH complementarity determining region 3 (CDR3) that includes an amino acid sequence at least 90%, 92%, 95%, 97% 98%, 99% or more identical to a sequence shown herein. The antibody binds to IL-6R, to IL-6R complexed with IL-6 (i.e., IL-6Rc) or both.

The three light chain CDRs include variable light chain (VL) CDR1 that includes an amino acid sequence at least 90%, 92%, 95%, 97% 98%, 99% or more identical to a sequence shown herein; a VL CDR2 that includes an amino acid sequence at least 90%, 92%, 95%, 97% 98%, 99% or more identical to the amino acid sequence shown herein; and a VL CDR3 that includes an amino acid sequence at least 90%, 92%, 95%, 97% 98%, 99% or more identical to a sequence shown herein. The antibody binds to IL-6R, to IL-6R complexed with IL-6 (i.e., IL-6Rc) or both.

In some embodiments, the combination therapy includes one or more fully human antibodies that bind to IL-6, IL-6R and/or IL-6Rc and prevent IL-6Rc from binding to gp130 such that gp130-mediated intracellular signaling cascade is not activated in the presence of these antibodies. Preferably, the antibodies have an affinity of at least 1×10⁻⁸ for IL-6, IL-6R, and/or IL-6Rc, and more preferably, the antibodies have an affinity of at least 1×10⁻⁹ for IL-6, IL-6R and/or IL-6Rc.

In some embodiments, combination therapies of the invention include an antibody that immunospecifically binds IL-6Rc wherein the antibody binds to an epitope that includes one or more amino acid residues on human IL-6 and/or human IL-6R. In some embodiments, the antibodies described herein bind to an epitope in domain 3 of IL-6 receptor (IL-6R). In some embodiments, the epitope to which the antibodies bind includes at least the amino acid sequence AERSKT (SEQ ID NO: 11).

Combination therapies of the invention also include fully human antibodies that specifically bind IL-6Rc, and antibodies that specifically bind both IL-6Rc and IL-6R, wherein the antibody exhibits greater than 50% inhibition of IL-6 mediated activation of the JAK/STAT pathway and MAPK cascade. For example, combination therapies of the invention exhibit greater than 55%, 60%, 65%, 70%, 75%, 80%, 85%, 90%, 92%, 95%, 97%, 98%, or 99% inhibition of IL-6 mediated functions including STAT3 activation, acute phase protein production, antibody production and cellular differentiation and/or proliferation.

In some embodiments, the antagonist of IL-6, IL-6R and/or the IL-6Rc is n mAb. In some embodiments, the antagonist of IL-6, IL-6R and/or IL-6Rc is a peptide, polypeptide or protein-based antagonist. In some embodiments, the antagonist IL-6, IL-6R and/or the IL-6Rc is a fusion protein. In some embodiments, the antagonist of IL-6, IL-6R and/or the IL-6Rc is a modified antibody antagonist or a non-antibody-based antagonist. Such antagonists include advanced antibody therapeutics, such as antibody fragments including, but not limited to, bispecific antibodies, Nanobodies® (as described in PCT Publication No. WO 08/071685, the contents of which are hereby incorporated by reference in their entirety, immunotoxins, and radio labeled therapeutics; peptide therapeutics; gene therapies, particularly intrabodies; oligonucleotide therapeutics such as aptamer therapeutics, antisense therapeutics, interfering RNA therapeutics; vaccines; and small molecules.

The combination therapies and methods of use thereof are preferably administered to human subjects. In some embodiments, the subject is non-responsive, less responsive over time, or has otherwise exhibited a decrease in responding to treatment with an antagonist of IL-6, IL-6R and/or the IL-6Rc, or is at risk for becoming non-responsive or less responsive to treatment with an antagonist IL-6, IL-6R and/or the IL-6Rc. The antagonist IL-6, IL-6R and/or the IL-6Rc to which the subject has become or is likely to become non-responsive or less responsive can be the same or a different antagonist IL-6, IL-6R and/or the IL-6Rc than the antagonist IL-6, IL-6R and/or the IL-6Rc to be administered in conjunction with a CD3 modulating agent.

In some embodiments, the autoimmune disease is RA, including forms of RA such as juvenile RA, or CD, including forms of CD such as luminal and fistulizing CD. In other embodiments, the autoimmune disease is selected from ankylosing spondylitis, asthma, Behcet's syndrome, glomerular nephritis, graft-versus-host disease, grave's disease, Hashimoto's thyroiditis, hidradenitis suppurativa, polyarticular juvenile arthritis, polymyositis/myositis/giant cell myocarditis and dermatomyositis, psoriasis, psoriatic arthritis, systemic lupus erythematosus (SLE), ulcerative colitis, undifferentiated polyarthritis, and uveitis.

In some embodiments, the CD3 modulating agent and the antagonist of IL-6, IL-6R and/or the IL-6Rc are present in the combination in an amount sufficient to produce a synergistic inhibitory effect on one or more biological activities of IL-6, IL-6R and/or the IL-6Rc in the subject. The CD3 modulators and antagonists of IL-6, IL-6R and/or the IL-6Rc can be prepared in separate formulations, or alternatively, they can be prepared in the same formulation. In embodiments where the CD3 modulator(s) and antagonist(s) of IL-6, IL-6R and/or the IL-6Rc are prepared in separate formulations, the CD3 modulator formulation(s) and antagonist of IL-6, IL-6R and/or the IL-6Rc formulation(s) can be administered simultaneously, or at separate times or intervals.

In some embodiments, the CD3 modulating agent is administered in a pharmaceutical formulation. In some embodiments, the CD3 modulating agent is an anti-CD3 antibody administered in a pharmaceutical formulation. Suitable pharmaceutical formulations are described, for example, in PCT Publication No. WO 07/033230, the contents of which are hereby incorporated by reference in their entirety.

In some embodiments, the CD3 modulating agent is an anti-CD3 antibody that is administered in a pharmaceutical formulation that includes a pH buffering agent in a range of 10 mM to 50 mM and effective in the range of 5.0 to 6.0, wherein said pH buffering agent is sodium acetate; sodium chloride in a range of 100 mM to 140 mM; 0.02% by weight/volume of a surfactant; and a pharmaceutically effective quantity of the anti-CD3 antibody. In some embodiments, the sodium chloride is 125 mM NaCl. In some embodiments, the surfactant is an ionic, anionic or zwitterionic surfactant. In some embodiments, the ionic surfactant is a polysorbate. In some embodiments, the pH buffering agent provides a pH range between 5.2 and 5.8. In some embodiments, the pH buffering agent provides a pH range between 5.4 and 5.6. In some embodiments, the pH buffering agent provides a pH of 5.5. In some embodiments, the surfactant is 0.02% by weight/volume and wherein the surfactant is polysorbate 80. In some embodiments, the pharmaceutically effective quantity of the anti-CD3 antibody is formulated to provide a quantity per dose in the range of 0.05 mg to 10 mg of anti-CD3 antibody. In some embodiments, the pharmaceutically effective quantity of the anti-CD3 antibody is formulated to provide a quantity per dose in the range of 0.1 mg to 5.0 mg of anti-CD3 antibody. In some embodiments, the pharmaceutically effective quantity of the anti-CD3 antibody is formulated to provide a quantity per dose in the range of 0.5 mg to 3.0 mg of anti-CD3 antibody. In some embodiments, the formulation is suitable for the intended route of administration such as, for example, intravenous, intradermal, subcutaneous; oral, inhalation, transdermal, transmucosal, or rectal administration.

In some embodiments, the pharmaceutical formulation for the anti-CD3 antibody consists essentially of a pH buffering agent in a range of 10 mM to 50 mM and effective in the range of 5.0 to 6.0, wherein said pH buffering agent is sodium acetate; sodium chloride in a range of 100 mM to 140 mM; 0.02% by weight/volume of a surfactant; and a pharmaceutically effective quantity of the anti-CD3 antibody. In some embodiments, the sodium chloride is 125 mM NaCl. In some embodiments, the surfactant is an ionic, anionic or zwitterionic surfactant. In some embodiments, the ionic surfactant is a polysorbate. In some embodiments, the pH buffering agent provides a pH range between 5.2 and 5.8. In some embodiments, the pH buffering agent provides a pH range between 5.4 and 5.6. In some embodiments, the pH buffering agent provides a pH of 5.5. In some embodiments, the surfactant is 0.02% by weight/volume and wherein the surfactant is polysorbate 80. In some embodiments, the pharmaceutically effective quantity of the anti-CD3 antibody is formulated to provide a quantity per dose in the range of 0.05 mg to 10 mg of anti-CD3 antibody. In some embodiments, the pharmaceutically effective quantity of the anti-CD3 antibody is formulated to provide a quantity per dose in the range of 0.1 mg to 5.0 mg of anti-CD3 antibody. In some embodiments, the pharmaceutically effective quantity of the anti-CD3 antibody is formulated to provide a quantity per dose in the range of 0.5 mg to 3.0 mg of anti-CD3 antibody. In some embodiments, the formulation is suitable for the intended route of administration such as, for example, intravenous, intradermal, subcutaneous; oral, inhalation, transdermal, transmucosal, or rectal administration.

In some embodiments, the pharmaceutical formulation for the anti-CD3 antibody consists essentially of a pH buffering agent comprising 25 mM sodium acetate effective in the range of 5.0 to 6.0; 125 mM sodium chloride; a surfactant comprising a polysorbate; and a pharmaceutically effective quantity of an anti-CD3 antibody. In some embodiments, the polysorbate is polysorbate 80. In some embodiments, the pH buffering agent provides a pH range between 5.2 and 5.8. In some embodiments, the pH buffering agent provides a pH range between 5.4 and 5.6. In some embodiments, the pH buffering agent provides a pH of 5.5. In some embodiments, the surfactant is 0.02% by weight/volume and wherein the surfactant is polysorbate 80. In some embodiments, the pharmaceutically effective quantity of the anti-CD3 antibody is formulated to provide a quantity per dose in the range of 0.05 mg to 10 mg of anti-CD3 antibody. In some embodiments, the pharmaceutically effective quantity of the anti-CD3 antibody is formulated to provide a quantity per dose in the range of 0.1 mg to 5.0 mg of anti-CD3 antibody. In some embodiments, the pharmaceutically effective quantity of the anti-CD3 antibody is formulated to provide a quantity per dose in the range of 0.5 mg to 3.0 mg of anti-CD3 antibody. In some embodiments, the formulation is suitable for the intended route of administration such as, for example, intravenous, intradermal, subcutaneous; oral, inhalation, transdermal, transmucosal, or rectal administration.

In a preferred embodiment, the formulation includes 25 mM sodium acetate, 125 mM sodium chloride, 0.02% by weight/volume of polysorbate 80, and a pH of 5.5. Preferably, the formulation is suitable for the intended route of administration such as, for example, intravenous, intradermal, subcutaneous; oral, inhalation, transdermal, transmucosal, or rectal administration.

The invention also provides methods of enhancing or otherwise supplementing anti-IL-6, anti-IL-6R and/or anti-IL-6Rc therapy in a subject that is receiving or has been administered an antagonist of IL-6, IL-6R and/or the IL-6Rc in an amount that is sufficient to produce a desired therapeutic outcome in the subject comprising administering to the subject a CD3 modulating agent. For example, the CD3 modulating agent is administered to the subject in an amount that is sufficient to reduce the dosage of antagonist of IL-6, IL-6R and/or the IL-6Rc that is needed to produce the desired therapeutic outcome in the subject. For example, the CD3 modulating agent is administered to the subject in an amount that is sufficient to decrease the frequency of administration of antagonist of IL-6, IL-6R and/or the IL-6Rc that is needed to produce the desired therapeutic outcome in the subject.

In some embodiments, the desired biological outcome is treating, delaying the progression of, preventing a relapse of, or alleviating a symptom of an autoimmune disease in the subject. In some embodiments, the autoimmune disease is RA including forms of RA such as juvenile RA, or the autoimmune disease is CD including forms of CD such as luminal and fistulizing CD. In some embodiments, the autoimmune disease is selected from the group consisting of ankylosing spondylitis, asthma, Behcet's syndrome, glomerular nephritis, graft-versus-host disease, grave's disease, Hashimoto's thyroiditis, hidradenitis suppurativa, polyarticular juvenile arthritis, polymyositis/myositis/giant cell myocarditis and dermatomyositis, psoriasis, psoriatic arthritis, SLE, ulcerative colitis, undifferentiated polyarthritis, and uveitis.

In some embodiments, the modulator of CD3 is an anti-CD3 antibody. For example, the anti-CD3 antibody is a monoclonal antibody. Suitable anti-CD3 antibodies for use in these methods include, for example, mouse, chimeric, humanized, domain and/or fully human monoclonal antibodies. In some embodiments, the anti-CD3 antibody is the fully human anti-CD3 monoclonal antibody NI-0401 comprising a heavy chain CDR1 having the amino acid sequence GYGMH (SEQ ID NO: 1), a heavy chain CDR2 having the amino acid sequence VIWYDGSKKYYVDSVKG (SEQ ID NO: 2), a heavy chain CDR3 having the amino acid sequence QMGYWHFDL (SEQ ID NO: 3), a light chain CDR1 having the amino acid sequence RASQSVSSYLA (SEQ ID NO: 4), a light chain CDR2 having the amino acid sequence DASNRAT (SEQ ID NO: 5), and a light chain CDR3 having the amino acid sequence QQRSNWPPLT (SEQ ID NO: 6). In some embodiments, the NI-0401 antibody further comprises a mutation in the heavy chain at an amino acid residue at position 234, 235, 265, or 297 or combinations thereof, and reduces the release of cytokines from a T-cell. In some embodiments, the mutation results in an alanine or glutamic acid residue at the position. In some embodiments, the NI-0401 antibody is an IgG1 isotype and contains at least a first mutation at position 234 and a second mutation at position 235, wherein the first mutation results in an alanine residue at position 234 and the second mutation results in a glutamic acid residue at position 235.

In a preferred embodiment, the subject is a human.

In some embodiments, the subject is non-responsive, less responsive or has exhibited a decrease in responding to treatment with an antagonist of IL-6, IL-6R and/or the IL-6Rc.

In some embodiments, the CD3 modulating agent is administered in an amount sufficient to produce a synergistic inhibitory effect on one or more biological activities of IL-6, IL-6R and/or IL-6Rc in the subject.

Also provided herein are uses of a combination of modulating agents for treating, delaying the progression of, preventing a relapse of, or alleviating a symptom of an autoimmune disease, wherein the combination of modulating agents includes a modulating agent that binds to CD3 and an antagonist that binds to IL-6, IL-6R and/or the IL-6Rc present in an amount sufficient to treat, delay the progression of, prevent a relapse of, or alleviate the symptom of the autoimmune disease in a subject. Also provided herein are uses of a combination of modulating agents in the manufacture of medicaments for treating, delaying the progression of, preventing a relapse of, or alleviating a symptom of an autoimmune disease, wherein the combination of modulating agents includes a modulating agent that binds to CD3 and an antagonist that binds to IL-6, IL-6R and/or the IL-6Rc present, and wherein the CD3 modulating agent and the antagonist of IL-6, IL-6R and/or the IL-6Rc are present in the medicament in an amount sufficient to treat, delay the progression of, prevent a relapse of, or alleviate the symptom of the autoimmune disease in a subject.

In some embodiments of these uses, the modulator of CD3 is an anti-CD3 antibody. For example, the anti-CD3 antibody is a monoclonal antibody, such as, e.g., a mouse, chimeric, humanized, domain or fully human monoclonal antibody. In some embodiments of these uses, the antagonist of IL-6, IL-6R and/or the IL-6Rc is an antibody or a fusion protein that binds to IL-6, IL-6R and/or the IL-6Rc. For example, the antibody that binds IL-6, IL-6R and/or the IL-6Rc is a monoclonal antibody such as, e.g., a chimeric, humanized, domain or fully human monoclonal antibody.

In some embodiments of these uses, the subject is a human. In some embodiments of these uses, the subject is non-responsive, less responsive or has stopped responding to treatment with an antagonist of IL-6, IL-6R and/or the IL-6Rc.

In some embodiments of these uses, the autoimmune disease is RA including forms of RA such as juvenile RA, or CD, including forms of CD such as luminal and fistulizing CD. In some embodiments, the autoimmune disease is selected from the group consisting of ankylosing spondylitis, asthma, Behcet's syndrome, glomerular nephritis, graft-versus-host disease, grave's disease, Hashimoto's thyroiditis, hidradenitis suppurativa, polyarticular juvenile arthritis, polymyositis/myositis/giant cell myocarditis and dermatomyositis, psoriasis, psoriatic arthritis, SLE, ulcerative colitis, undifferentiated polyarthritis, and uveitis.

In some embodiments of these uses, the CD3 modulating agent and the antagonist of IL-6, IL-6R and/or the IL-6Rc are present in the combination of modulating agents in an amount sufficient to produce a synergistic inhibitory effect on one or more biological activities of IL-6, IL-6R and/or IL-6Rc in the subject.

In some embodiments of these uses, the CD3 modulating agent is a fully human anti-CD3 monoclonal antibody that includes a heavy chain CDR1 having the amino acid sequence GYGMH (SEQ ID NO: 1), a heavy chain CDR2 having the amino acid sequence VIWYDGSKKYYVDSVKG (SEQ ID NO: 2), a heavy chain CDR3 having the amino acid sequence QMGYWHFDL (SEQ ID NO: 3), a light chain CDR1 having the amino acid sequence RASQSVSSYLA (SEQ ID NO: 4), a light chain CDR2 having the amino acid sequence DASNRAT (SEQ ID NO: 5), and a light chain CDR3 having the amino acid sequence QQRSNWPPLT (SEQ ID NO: 6).

In some embodiments of these uses, the anti-CD3 antibody also includes a mutation in the heavy chain at an amino acid residue at position 234, 235, 265, or 297 or combinations thereof, and reduces the release of cytokines from a T-cell. For example, the mutation results in an alanine or glutamic acid residue at an amino acid residue at position 234, 235, 265, or 297 or combinations thereof. In some embodiments of these uses, the anti-CD3 antibody is an IgG1 isotype and contains at least a first mutation at position 234 and a second mutation at position 235, wherein the first mutation results in an alanine residue at position 234 and the second mutation results in a glutamic acid residue at position 235.

Also provided herein are uses of an CD3 modulating agent for enhancing or supplementing anti-IL-6, anti-IL-6R and/or anti-IL-6Rc therapy in a subject that is receiving or has been administered an antagonist of IL-6, IL-6R and/or the IL-6Rc in an amount that is sufficient to produce a desired therapeutic outcome in the subject. In these uses, the subject is currently receiving or has received in the past an anti-IL-6, anti-IL-6R and/or anti-IL-6Rc therapy to achieve a desired therapeutic outcome, e.g., treating, delaying the progression of, preventing a relapse of, or alleviating a symptom of an autoimmune disease in the subject. In some embodiments of these uses, the subject is non-responsive, less responsive or otherwise exhibits a decrease in responsiveness to the anti-IL-6, anti-IL-6R and/or anti-IL-6Rc therapy. In some embodiments of these uses, the CD3 modulating agent is used in an amount that is sufficient to reduce the dosage of antagonist of IL-6, IL-6R and/or the IL-6Rc that is needed to produce the desired therapeutic outcome in the subject. In some embodiments of these uses, the CD3 modulating agent is used in an amount that is sufficient to decrease the frequency of administration of antagonist of IL-6, IL-6R and/or the IL-6Rc that is needed to produce the desired therapeutic outcome in the subject.

Also provided are uses of an CD3 modulating agent in the manufacture of a medicament for enhancing or supplementing anti IL-6, anti-IL-6R and/or anti-IL-6Rc therapy in a subject that is receiving or has been administered an antagonist of IL-6, IL-6R and/or the IL-6Rc in an amount that is sufficient to produce a desired therapeutic outcome in the subject. In these uses, the subject is currently receiving or has received in the past an anti IL-6, anti-IL-6R and/or anti-IL-6Rc therapy to achieve a desired therapeutic outcome, e.g., treating, delaying the progression of, preventing a relapse of, or alleviating a symptom of an autoimmune disease in the subject. In some embodiments of these uses, the subject is non-responsive, less responsive or otherwise exhibits a decrease in responsiveness to the anti IL-6, anti-IL-6R and/or anti-IL-6Rc therapy. In some embodiments of these uses, the CD3 modulating agent is present in the medicament in an amount that is sufficient to reduce the dosage of antagonist of IL-6, IL-6R and/or the IL-6Rc that is needed to produce the desired therapeutic outcome in the subject. In some embodiments of these uses, the CD3 modulating agent is present in the medicament in an amount that is sufficient to decrease the frequency of administration of antagonist of IL-6, IL-6R and/or the IL-6Rc that is needed to produce the desired therapeutic outcome in the subject.

In some embodiments of these uses, the modulator of CD3 is an anti-CD3 antibody. For example, the anti-CD3 antibody is a monoclonal antibody, such as, e.g., a mouse, chimeric, humanized, domain or fully human monoclonal antibody. In some embodiments of these uses, the antagonist of IL-6, IL-6R and/or the IL-6Rc is an antibody or a fusion protein that binds to IL-6, IL-6R and/or the IL-6Rc. For example, the anti IL-6, anti-IL-6R and/or anti-IL-6Rc antibody is a monoclonal antibody such as, e.g., a chimeric, humanized, domain or fully human monoclonal antibody.

In some embodiments of these uses, the subject is a human. In some embodiments of these uses, the subject is non-responsive, less responsive or has stopped responding to treatment with an antagonist of IL-6, IL-6R and/or the IL-6Rc.

In some embodiments of these uses, the subject is currently receiving or has received in the past an anti IL-6, anti-IL-6R and/or anti-IL-6Rc therapy to achieve a desired level of treating, delaying the progression of, preventing a relapse of, or alleviating a symptom of an autoimmune disease in the subject. In some embodiments of these uses, the autoimmune disease is RA, including forms of RA such as juvenile RA, or CD, including forms of CD such as luminal and fistulizing CD. In some embodiments, the autoimmune disease is selected from the group consisting of ankylosing spondylitis, asthma, Behcet's syndrome, glomerular nephritis, graft-versus-host disease, grave's disease, Hashimoto's thyroiditis, hidradenitis suppurativa, polyarticular juvenile arthritis, polymyositis/myositis/giant cell myocarditis and dermatomyositis, psoriasis, psoriatic arthritis, SLE, ulcerative colitis, undifferentiated polyarthritis, and uveitis.

In some embodiments of these uses, the CD3 modulating agent and the antagonist of IL-6, IL-6R and/or the IL-6Rc are used and/or are present in the combination of modulating agents in an amount sufficient to produce a synergistic inhibitory effect on one or more biological activities of IL-6, IL-6R and/or the IL-6Rc in the subject.

In some embodiments of these uses, the CD3 modulating agent is a fully human anti-CD3 monoclonal antibody that includes a heavy chain CDR1 having the amino acid sequence GYGMH (SEQ ID NO: 1), a heavy chain CDR2 having the amino acid sequence VIWYDGSKKYYVDSVKG (SEQ ID NO: 2), a heavy chain CDR3 having the amino acid sequence QMGYWHFDL (SEQ ID NO: 3), a light chain CDR1 having the amino acid sequence RASQSVSSYLA (SEQ ID NO: 4), a light chain CDR2 having the amino acid sequence DASNRAT (SEQ ID NO: 5), and a light chain CDR3 having the amino acid sequence QQRSNWPPLT (SEQ ID NO: 6).

In some embodiments of these uses, the anti-CD3 antibody also includes a mutation in the heavy chain at an amino acid residue at position 234, 235, 265, or 297 or combinations thereof, and reduces the release of cytokines from a T-cell. For example, the mutation results in an alanine or glutamic acid residue at an amino acid residue at position 234, 235, 265, or 297 or combinations thereof. In some embodiments of these uses, the anti-CD3 antibody is an IgG1 isotype and contains at least a first mutation at position 234 and a second mutation at position 235, wherein the first mutation results in an alanine residue at position 234 and the second mutation results in a glutamic acid residue at position 235.

The present invention also provides methods of treating or preventing pathologies associated with aberrant IL-6 receptor activation and/or aberrant IL-6 signaling (cis and/or trans) or alleviating a symptom associated with such pathologies, by administering a combination therapy of the invention to a subject in which such treatment or prevention is desired. The subject to be treated is, e.g., human. The combination therapy is administered in an amount sufficient to treat, prevent or alleviate a symptom associated with the pathology. The amount of combination therapy sufficient to treat or prevent the pathology in the subject is, for example, an amount that is sufficient to reduce IL-6Rc induced activation of the JAK/STAT pathway or MAPK cascade. For example, IL-6Rc induced activation of the JAK/STAT pathway or MAPK cascade is decreased when the level of STAT3 activation in the presence of a monoclonal antibody of the invention is greater than or equal to 5%, 10%, 20%, 25%, 30%, 40%, 50%, 60%, 70%, 75%, 80%, 90%, 95%, 99%, or 100% lower than a control level of STAT3 activation (i.e., the level of STAT3 activation in the absence of the monoclonal antibody). Those skilled in the art will appreciate that the level of STAT3 activation can be measured using a variety of assays, including, for example, commercially available ELISA kits.

Pathologies treated and/or prevented using the combination therapies of the invention (e.g., fully human monoclonal antibody) include, for example, sepsis, cancer (e.g., multiple myeloma disease (MM), renal cell carcinoma (RCC), plasma cell leukaemia, lymphoma, B-lymphoproliferative disorder (BLPD), and prostate cancer), bone resorption, osteoporosis, cachexia, psoriasis, mesangial proliferative glomerulonephritis, Kaposi's sarcoma, AIDS-related lymphoma, and inflammatory diseases (e.g., RA, systemic onset juvenile idiopathic arthritis, hypergammaglobulinemia, CD, ulcerative colitis, systemic lupus erythematosus (SLE), multiple sclerosis, Castleman's disease, IgM gammopathy, cardiac myxoma, asthma, allergic asthma and autoimmune insulin-dependent diabetes mellitus).

Pharmaceutical compositions according to the invention can include a modulator of CD3 and an antagonist of IL-6, IL-6R and/or the IL-6Rc and a carrier. These pharmaceutical compositions can be included in kits, such as, for example, diagnostic kits.

One skilled in the art will appreciate that the combination therapies of the invention have a variety of uses. For example, the combination therapies of the invention are used as therapeutic agents to prevent IL-6 receptor activation in disorders such as, for example, sepsis, cancer (e.g., multiple myeloma disease (MM), renal cell carcinoma (RCC), plasma cell leukaemia, lymphoma, B-lymphoproliferative disorder (BLPD), and prostate cancer), bone resorption, osteoporosis, cachexia, psoriasis, mesangial proliferative glomerulonephritis, Kaposi's sarcoma, AIDS-related lymphoma, and inflammatory diseases (e.g., RA, systemic onset juvenile idiopathic arthritis, hypergammaglobulinemia, CD, ulcerative colitis, systemic lupus erythematosus (SLE), multiple sclerosis, Castleman's disease, IgM gammopathy, cardiac myxoma, asthma, allergic asthma and autoimmune insulin-dependent diabetes mellitus). The combination therapies of the invention are also used as reagents in diagnostic kits or as diagnostic tools, or these antibodies can be used in competition assays to generate therapeutic reagents.

### BRIEF DESCRIPTION OF THE DRAWINGS

FIG. 1 is a graph depicting that i.p. injection of anti-CD3 (clone 145-2C11) ameliorates for a short period of time (4-5 days), while i.p. treatment with an anti-IL-6 (clone MP5-20F3) antibody alone does not ameliorate arthritis in mice with collagen induced arthritis (CIA). In contrast the combitherapy (i.e., combination therapy) controls arthritis in mice with CIA for prolonged time.

### DETAILED DESCRIPTION

The present invention provides combination therapies and methods that use modulators of CD3 and antagonists of IL-6, IL-6R and/or the IL-6Rc to treat, delay the progression of, prevent relapse of or alleviate a symptom of an autoimmune disease.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the terms Interleukin-6 Receptor, IL-6R, Interleukin-6 Receptor-alpha, IL-6Rα, cluster differentiation factor 126, and CD126 are synonymous and are used interchangeably. Each term refers to the homodimeric protein, except as otherwise indicated.

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, domain antibody, F_{ab}, F_{ab}, and F_{(ab′)2} fragments, and an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., 2nd ed. Raven Press, N.Y. (1989)). The variable regions of each light/heavy chain pair form the antibody binding site.

The term “monoclonal antibody” and the abbreviation mAb, or the term “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the mAb are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia &Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM; preferably ≦100 nM and most preferably ≦10 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides are quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to a CD3 epitope when the equilibrium binding constant (K_{d}) is ≦1 μM, preferably ≦100 nM, more preferably ≦10 nM, and most preferably ≦100 pM to about 1 pM, as measured by assays such as radioligand binding assays or similar assays known to those skilled in the art.

Those skilled in the art will recognize that it is possible to determine, without undue experimentation, if a mAb has the same specificity as a mAb used in the combination therapies of the invention (e.g., mAb NI-0401) by ascertaining whether the former prevents the latter from binding to a antigen polypeptide (e.g., CD3, IL-6, IL-6R and/or IL-6Rc). If the mAb being tested competes with a mAb used in the combination therapies of the invention, as shown by a decrease in binding by the mAb of the invention, then the two mAbs bind to the same, or a closely related, epitope. Another way to determine whether a mAb has the specificity of a mAb used in the combination therapies of the invention is to pre-incubate the mAb of the invention with the antigen polypeptide (e.g., CD3, IL-6, IL-6R and/or IL-6Rc) with which it is normally reactive, and then add the mAb being tested to determine if the mAb being tested is inhibited in its ability to bind the antigen polypeptide. If the mAb being tested is inhibited then, in all likelihood, it has the same, or functionally equivalent, epitopic specificity as the mAb used in the combination therapies of the invention.

Various procedures known within the art are used for the production of the mAbs directed against a protein such as a CD3, IL-6, IL-6R and/or IL-6Rc protein, or against derivatives, fragments, analogs homologs or orthologs thereof. (See, e.g., Antibodies: A Laboratory Manual, Harlow E, and Lane D, 1988, Cold Spring Harbor Laboratory Press, Cold Spring Harbor, N.Y., incorporated herein by reference). Fully human antibodies are antibody molecules in which the entire sequence of both the light chain and the heavy chain, including the CDRs, arise from human genes. Such antibodies are termed “human antibodies” or “fully human antibodies” herein. Human mAbs are prepared, for example, using the procedures described in PCT Publication No. WO 05/118635. Human mAbs can be also prepared by using trioma technique; the human B-cell hybridoma technique (see Kozbor, et al., 1983 Immunol Today 4:72); and the EBV hybridoma technique to produce human mAbs (see Cole, et al., 1985 In: MONOCLONAL ANTIBODIES AND CANCER THERAPY, Alan R. Liss, Inc., pp. 77-96). Human mAbs may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Antibodies are purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

It is desirable to modify the antibody used in the combination therapies of the invention with respect to effector function, so as to enhance, e.g., the effectiveness of the antibody in treating immune-related diseases. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

Those of ordinary skill in the art will recognize that a large variety of possible moieties can be coupled to the modulating agents, antagonists and antibodies used in the combination therapies and methods provided herein. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling is accomplished by any chemical reaction that will bind the two molecules so long as the modulating agent, antagonist or the antibody and the other moiety retain their respective activities. This linkage can include many chemical mechanisms, for instance covalent binding, affinity binding, intercalation, coordinate binding and complexation. The preferred binding is, however, covalent binding. Covalent binding is achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the human heavy chain immunoglobulin molecules and the human light chain immunoglobulin molecules shown herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The following terms are used to describe the relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window,” as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman PNAS (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long′ more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to CD3, under suitable binding conditions, (2) ability to block appropriate CD3 binding, or (3) ability to inhibit CD3-expressing cell growth in vitro or in vivo. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

### Antibodies

Exemplary anti-CD3 antibodies for use in the combination therapies provided herein include those antibodies described in PCT Publication No. WO 05/118635, the contents of which are hereby incorporated by reference in their entirety, or an anti-CD3 antibody that binds to the same epitope as those antibodies described in PCT Publication No. WO 05/118635. Other suitable anti-CD3 mAbs for use in the combination therapies and methods provided herein include, but are not limited to, Orthoclone OKT3 (also known as Muromonab), human OKT3γ1 (HOKT3γ1, also known as Teplizumab), ChAglyCD3 (also known as Otelixizumab) and Nuvion® (also known as Visilizumab), or antibodies that bind to the same epitope as Orthoclone OKT3, human OKT3γ1 (HOKT3γ1), ChAglyCD3 or Nuvion® (Visilizumab).

Suitable anti-IL-6, anti-IL-6R and/or anti-IL-6Rc antibodies for use in the combination therapies of the invention include the antibodies described in PCT/US2009/043734, filed May 13, 2009 and published as WO 2009/140348, the contents of which are hereby incorporated by reference in their entirety, such as, for example, the 39B9 VL1 antibody, the 39B9 VL5 antibody, the 12A antibody, and the 5C antibody. These antibodies show specificity for human IL-6Rc and/or both IL-6Rc and IL-6R and they have been shown to inhibit the functional activity of IL-6Rc (i.e., binding to gp130 to induce the signaling cascade) in vitro.

Suitable anti-IL-6, anti-IL-6R and/or anti-IL-6Rc antibodies for use in the combination therapies of the invention include the antibodies described in U.S. Pat. No. 5,670,373, U.S. Pat. No. 5,888,510, PCT Publication No. WO 08/065384, PCT Publication No. WO 08/065378, PCT Publication No. WO 08/019061, PCT Publication No. WO 07/143168, the contents of which are hereby incorporated by reference in their entirety.

Also included in the invention are antibodies that bind to the same epitope as the antibodies described herein. For example, combination therapies of the invention include antibodies that specifically bind to IL-6R, wherein the antibody binds to an epitope that includes one or more amino acid residues on human IL-6R (e.g., GenBank Accession No. P08887). Combination therapies of the invention include antibodies that specifically bind IL-6Rc, wherein the antibody binds to an epitope that includes one or more amino acid residues on human IL-6 (e.g., GenBank Accession No. NP_{—}000591), IL-6R (e.g., GenBank Accession No. P08887), or both.

Those skilled in the art will recognize that it is possible to determine, without undue experimentation, if a monoclonal antibody (e.g., fully human monoclonal antibody) has the same specificity as a monoclonal antibody used in the combination therapies of the invention by ascertaining whether the former prevents the latter from binding to IL-6, IL-6R, IL-6Rc and/or gp130. If the monoclonal antibody being tested competes with the monoclonal antibody used in the combination therapies of the invention, as shown by a decrease in binding by the monoclonal antibody used in the combination therapies of the invention, then the two monoclonal antibodies bind to the same, or a closely related, epitope.

Therapeutic Administration and Formulations

It will be appreciated that administration of combinations of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Combination therapies of the invention, which include a CD3 modulating agent and an antagonist of IL-6, IL-6R and/or the IL-6Rc, are used to treat or alleviate a symptom associated with an immune-related disorder, such as, for example, an autoimmune disease.

Autoimmune diseases include, for example, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barré syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Ménière's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Diseases or disorders related to aberrant IL-6 signaling include sepsis, cancer (e.g., multiple myeloma disease (MM), renal cell carcinoma (RCC), plasma cell leukaemia, lymphoma, B-lymphoproliferative disorder (BLPD), and prostate cancer), bone resorption, osteoporosis, cachexia, psoriasis, mesangial proliferative glomerulonephritis, Kaposi's sarcoma, AIDS-related lymphoma, and inflammatory diseases (e.g., rheumatoid arthritis, systemic onset juvenile idiopathic arthritis, hypergammaglobulinemia, Crohn's disease, ulcerative colitis, systemic lupus erythematosus (SLE), multiple sclerosis, Castleman's disease, IgM gammopathy, cardiac myxoma, asthma, allergic asthma and autoimmune insulin-dependent diabetes mellitus).

Symptoms associated with immune-related disorders include, for example, inflammation, fever, loss of appetite, weight loss, abdominal symptoms such as, for example, abdominal pain, diarrhea or constipation, joint pain or aches (arthralgia), fatigue, rash, anemia, extreme sensitivity to cold (Raynaud's phenomenon), muscle weakness, muscle fatigue, changes in skin or tissue tone, shortness of breath or other abnormal breathing patterns, chest pain or constriction of the chest muscles, abnormal heart rate (e.g., elevated or lowered), light sensitivity, blurry or otherwise abnormal vision, and reduced organ function. For example, symptoms of RA include joint pain, joint tenderness, joint swelling, fatigue, loss of appetite, joint stiffness including morning stiffness lasting more than 1 hour, widespread muscle aches, weakness, anemia (e.g., due to failure of the bone marrow to produce sufficient new red blood cells), eye burning, itching, and other discharge, deformities in the hands and feet, limited range of motion, low-grade fever, lung inflammation (pleurisy), nodules under the skin, numbness or tingling sensation, skin redness, paleness, warmth or inflammation, and swollen glands. Symptoms of CD include abdominal cramps and pain, fever, fatigue, loss of appetite, pain associated with passing stool (tenesmus), persistent, watery diarrhea, unintentional weight loss, constipation, eye inflammation, fistulas, joint pain, liver inflammation, mouth ulcers, rectal bleeding, skin rash and swollen gums.

The therapeutic combinations of CD3 modulators and antagonists of IL-6, IL-6R and/or IL-6Rc are administered to a subject suffering from an immune-related disorder, such as an autoimmune disease or an inflammatory disorder, such as, for example, RA and CD. A subject suffering from an autoimmune disease or an inflammatory disorder is identified by methods known in the art. For example, subjects suffering from an autoimmune disease such as RA or CD, are identified using any of a variety of clinical and/or laboratory tests such as, physical examination, radiologic examination and blood, urine and stool analysis to evaluate immune status. Patients suffering from CD are identified, e.g., using an upper gastrointestinal (GI) series and/or a colonoscopy to evaluate the small and large intestines, respectively. Patients suffering from RA are identified, e.g., using blood tests to distinguish RA from other types of arthritis, e.g., the anti-CCP antibody test, a complete blood count, a C-reactive protein test, evaluation of erythrocyte sedimentation rate, joint x-rays, ultrasound or MRI, rheumatoid factor test, and synovial fluid analysis.

Administration of the therapeutic combinations of CD3 modulators and antagonists of IL-6, IL-6R and/or IL-6Rc to a patient suffering from an autoimmune disease or an inflammatory disorder is considered successful if any of a variety of laboratory or clinical results is achieved. For example, administration of the therapeutic combinations of CD3 modulators and antagonists of IL-6, IL-6R and/or IL-6Rc to a patient suffering from an immune-related disorder such as an autoimmune disease or an inflammatory disorder, such as, for example, RA or CD, is considered successful one or more of the symptoms associated with the disorder is alleviated, reduced, inhibited or does not progress to a further, i.e., worse, state. Administration of the therapeutic combinations of CD3 modulators and antagonists of IL-6, IL-6R and/or IL-6Rc to a patient suffering from an immune-related disorder such as an autoimmune disease or an inflammatory disorder is considered successful if the disorder, e.g., an autoimmune disorder, enters remission or does not progress to a further, i.e., worse, state.

In some embodiments, the combination therapies used to treat an autoimmune disease are administered in combination with any of a variety of known anti-inflammatory and/or immunosuppressive compounds. Suitable known compounds include, but are not limited to methotrexate, cyclosporin A (including, for example, cyclosporin microemulsion), tacrolimus, corticosteroids, statins, interferon beta, non-steroidal anti-inflammatory agents, 6-MP (Mercaptopurine, also called 6-Mercaptopurine, or Purinethol). For example, subjects with RA are also administered a disease modifying anti-rheumatic drug (DMARD) such as methotrexate or leflunomide; an anti-inflammatory medication such as aspirin or a nonsteroidal anti-inflammatory drug (NSAID), an anti-malarial medication such as hydroxychloroquine or sulfasalazine, alone or in further combination with methotrexate; a corticosteroid, a cyclooxygenase-2 (COX-2) inhibitor, a specific white blood cell modulating biological agent to control inflammation such as, e.g., abatacept or rituximab, and combinations thereof. For example, subjects with CD are also administered an anti-diarrheal drug such as loperamide or other over the counter medications, an aminosalicylate (5-ASA) to control inflammation, a corticosteroid such as prednisone or methylprednisolone, an immunomodulator such as azathioprine or 6-mercaptopurine, an antibiotic, or combinations thereof.

In some embodiments, the combination therapies used to treat an autoimmune disease are used in conjunction with a surgical method of treating or otherwise alleviating the autoimmune disease. For example, subjects with RA may require surgery to correct severely affected joints, relieve joint pain, correct deformities, and improve joint function. Subjects with CD may require surgery such as a bowel resection or other surgical methods to reduce bleeding or other hemorrhage, to remove fistulas, to treat infections and abscesses, or to correct intestinal narrowing and strictures.

The combinations of modulating agents, e.g., CD3 modulators and antagonists of IL-6, IL-6R and/or IL-6Rc, are administered to a subject in an amount sufficient to have a desired modulation effect due to binding with the respective targets. In some embodiments, administration of the combinations will abrogate or inhibit or otherwise interfere with at least one biological property and/or biological activity of that target, such as e.g., a signaling function of the target, binding of the target with an endogenous ligand to which it naturally binds, etc.

A therapeutically effective amount of a combination described herein relates generally to the amount needed to achieve a therapeutic objective such as, for example, treating, delaying the progression of, preventing a relapse of, or alleviating a symptom of an autoimmune disease. As noted above, this may be a binding interaction between the antibody and its target antigen(s) that, in certain cases, interferes with the functioning of the target(s). The amount required to be administered will furthermore depend on the binding affinity of the antibody for its specific antigen(s), and will also depend on the rate at which an administered antibody is depleted from the free volume of the subject to which it is administered. Common ranges for therapeutically effective dosing of an antibody, antibody combination or antibody fragment described herein may be, by way of nonlimiting example, from about 0.1 mg/kg body weight to about 50 mg/kg body weight. Common dosing frequencies may range, for example, from twice daily to once a week.

Efficaciousness of treatment is determined in association with any known method for diagnosing or treating the particular inflammatory-related disorder. Alleviation of one or more symptoms of the inflammatory-related disorder indicates that the antibody confers a clinical benefit.

The CD3 modulators and antagonists of IL-6, IL-6R and/or IL-6Rc can be prepared in separate formulations, or alternatively, they can be prepared in the same formulation. In embodiments where the CD3 modulator(s) and the antagonist(s) IL-6, IL-6R and/or IL-6Rc are prepared in separate formulations, the CD3 modulator formulation(s) and the antagonist of IL-6, IL-6R and/or IL-6Rc formulation(s) can be administered simultaneously, or at separate times or intervals.

Combination therapies of the invention are formulated to be compatible with the intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples, including the experiments conducted and results achieved are provided for illustrative purposes only and are not to be construed as limiting upon the present invention.

Anti-Mouse IL-6 mAb:

A neutralizing anti-mouse IL-6 mAb, clone MP5-20F3, has been described. (Starnes H F Jr et al; Anti-IL-6 monoclonal antibodies protect against lethal Escherichia coli infection and lethal tumor necrosis factor-alpha challenge in mice. (1990) J Immunol, 145: 4185).

Anti-Mouse CD3 mAb:

A hamster mAb, 145-2C11, directed against the epsilon chain of the mouse CD3/TcR complex, has been described. (Leo O et al; Identification of a monoclonal antibody specific for a murine T3 polypeptide. (1987) PNAS. 84: 1374).

Collagen Induced Arthritis (CIA):

CIA was induced in male DBA/1 mice, by immunization with 100 μg of bovine type II collagen emulsified in Freund's complete adjuvant (CFA). Three weeks later, a booster injection consisting of 100 μg of collagen in Freund's incomplete adjuvant (IFA) was performed. The mice developed a classic course of disease characterized by chronic inflammation in the limbs and joints commencing a few days after the antigenic boost.

Neutralizing IL-6 in CIA:

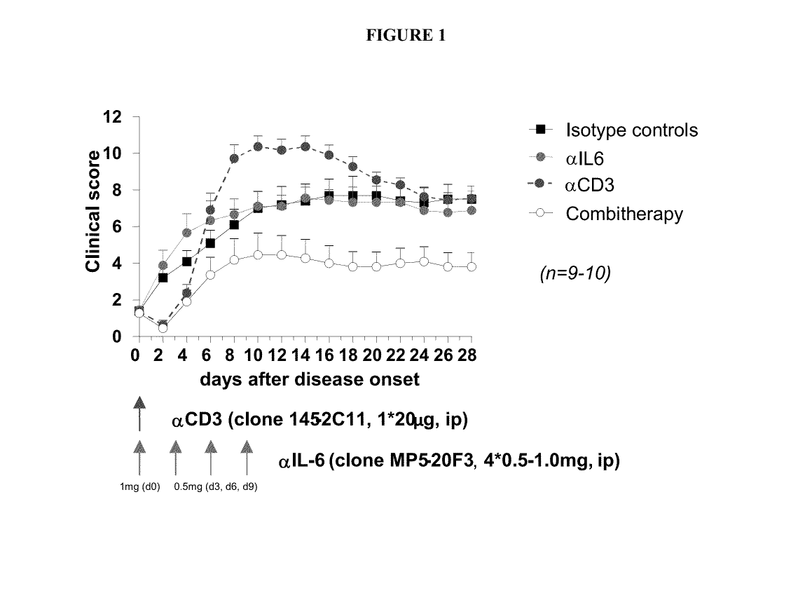

The effect of the neutralizing anti-IL6 antibody MP5-20F3 in CIA used in a prophylactic manner has been published. (Liang B et al; Evaluation of anti-IL-6 monoclonal antibody therapy using murine type II collagen-induced arthritis. (2009) Journal of Inflammation, 6:10). Prophylactic treatment with the antibody reduced the incidence and severity of arthritis compared to control mAb treated mice. The data shown in FIG. 1 (filled circles, solid line) demonstrate that treatment with MP5-20F3 does not reduce the severity of arthritis.

Targeting CD3 in CIA:

To address the effect of anti-CD3 therapy in arthritis, CIA mice were treated with 145-2C11 at onset of disease with a protocol previously described (Notley C A et al; Anti-CD3 therapy expands the numbers of CD4⁺ and CD8⁺ Treg cells and induces sustained amelioration of collagen-induced arthritis. (2010) Arthritis & Rheumatism 62: 171). The data shown in FIG. 1 (filled circles, dotted line) confirms that a single i.p. dose of 20 μg of 145-2C11 at disease onset ameliorates the severity of the disease but for a limited period (i.e. 4-5 days).

Neutralizing IL-6 while Targeting T Cells with an Anti-CD3 mAb in CIA:

The results, shown in FIG. 1, demonstrate that the combination approach is significantly more effective in preventing disease evolution as well as prolonging time to relapse than occurs with anti-IL-6 or anti-CD3 mAb therapy alone. These experiments establish, for the first time, that IL-6 and T cell responses cooperate on a long term basis during the chronic phase of an autoimmune disease.

Combination Therapies Using Anti-CD3 mAb and Anti-IL-6 mAbs:

Modifying components of both innate and acquired immunity leads to disease amelioration in a model of autoimmunity. Combination therapy with two mAbs that target CD3 on T cells and neutralize IL-6 produces a potent synergy that reduces disease severity and prevents disease relapse. This data thus provides the basis to support using such a combination strategy to obtain an effective long-term treatment for RA, CD.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating, delaying the progression of, preventing a relapse of, or alleviating a symptom of an autoimmune disease, the method comprising administering a combination of modulating agents to a subject in need thereof in an amount sufficient to treat, delay the progression of, prevent a relapse of, or alleviate the symptom of the autoimmune disease in the subject, wherein said combination of modulating agents comprises a modulating agent that binds to CD3 and an antagonist that binds to IL-6, IL-6R and/or IL-6Rc.

2. The method of claim 1, wherein the modulator of CD3 is an anti-CD3 antibody.

3. The method of claim 2, wherein the anti-CD3 antibody is a monoclonal antibody.

4. The method of claim 2, wherein the anti-CD3 antibody is a mouse, chimeric, humanized, domain or fully human monoclonal antibody.

5. The method of claim 1, wherein the antagonist of IL-6, IL-6R and/or IL-6Rc is an anti IL-6, anti-IL-6R and/or anti-IL-6Rc antibody.

6. The method of claim 5, wherein the anti IL-6, anti-IL-6R and/or anti-IL-6Rc antibody is a monoclonal antibody.

7. The method of claim 5, wherein the anti IL-6, anti-IL-6R and/or anti-IL-6Rc antibody is a chimeric, humanized, domain or fully human monoclonal antibody.

8. The method of claim 1, wherein the antagonist of IL-6, IL-6R and/or IL-6Rc is soluble gp130.

9. The method of claim 1, wherein the subject is a human.

10. The method of claim 1, wherein the autoimmune disease is rheumatoid arthritis.

11. The method of claim 1, wherein the autoimmune disease is Crohn's disease.

12. The method of claim 1, wherein the autoimmune disease is selected from the group consisting of ankylosing spondylitis, asthma, Behcet's syndrome, glomerular nephritis, graft-versus-host disease, grave's disease, Hashimoto's thyroiditis, hidradenitis suppurativa, juvenile rheumatoid arthritis, luminal and fistulizing Crohn's disease, polyarticular juvenile arthritis, polymyositis/myositis/giant cell myocarditis and dermatomyositis, psoriasis, psoriatic arthritis, rheumatoid arthritis, systemic lupus erythematosus, ulcerative colitis, undifferentiated polyarthritis, and uveitis.

13. The method of claim 1, wherein the CD3 modulating agent and the antagonist of IL-6, IL-6R and/or the IL-6Rc are present in the combination in an amount sufficient to produce a synergistic inhibitory effect on one or more biological activities of IL-6, IL-6R and/or IL-6Rc in said subject.

14. The method of claim 4, wherein the anti-CD3 antibody is a fully human anti-CD3 monoclonal antibody comprising a heavy chain CDR1 having the amino acid sequence GYGMH (SEQ ID NO: 1), a heavy chain CDR2 having the amino acid sequence VIWYDGSKKYYVDSVKG (SEQ ID NO: 2), a heavy chain CDR3 having the amino acid sequence QMGYWHFDL (SEQ ID NO: 3), a light chain CDR1 having the amino acid sequence RASQSVSSYLA (SEQ ID NO: 4), a light chain CDR2 having the amino acid sequence DASNRAT (SEQ ID NO: 5), and a light chain CDR3 having the amino acid sequence QQRSNWPPLT (SEQ ID NO: 6).

15. The method of claim 14, wherein the antibody further comprises a mutation in the heavy chain at an amino acid residue at position 234, 235, 265, or 297 or combinations thereof, and reduces the release of cytokines from a T-cell.

16. The method of claim 15, wherein said mutation results in an alanine or glutamic acid residue at said position.

17. The method of claim 16, wherein the antibody is an IgG1 isotype and contains at least a first mutation at position 234 and a second mutation at position 235, wherein said first mutation results in an alanine residue at position 234 and said second mutation results in a glutamic acid residue at position 235.

18. The method of claim 14, wherein the antibody further comprises a variable heavy chain region comprising the amino acid sequence of QVQLVESGGGVVQPGRSLRLSCAASGFKFS GYGMHWVRQAPGKGLEWVAVIWYDGSKKYYVDSVKGRFTISRDNSKNTLYLQMNSL RAEDTAVYYCARQMGYWHFDLWGRGTLVTVSS (SEQ ID NO: 8) and a variable light chain region comprising the amino acid sequence of EIVLTQSPATLSLSPGERATLSCRASQ SVSSYLAWYQQKPGQAPRLLIYDASNRATGIPARFSGSGSGTDFTLTISSLEPEDFAVYY CQQRSNWPPLTFGGGTKVEIK (SEQ ID NO: 10).


SEQUENCE LISTING (20 sequences)

SEQ ID NO: 1 - protein, 5 aa, Homo sapiens
GYGMH

SEQ ID NO: 2 - protein, 17 aa, Homo sapiens
VIWYDGSKKYYVDSVKG

SEQ ID NO: 3 - protein, 9 aa, Homo sapiens
QMGYWHFDL

SEQ ID NO: 4 - protein, 11 aa, Homo sapiens
RASQSVSSYLA

SEQ ID NO: 5 - protein, 7 aa, Homo sapiens
DASNRAT

SEQ ID NO: 6 - protein, 10 aa, Homo sapiens
QQRSNWPPLT

SEQ ID NO: 7 - DNA, 354 bp, Homo sapiens
caggtgcagctggtggagtccgggggaggcgtggtccagcctgggaggtccctgagactctcctgtgcagcgtctggattcaagttcagtggctatggcatgcactgggtccgccaggctccaggcaaggggctggagtgggtggcagttatatggtatgatggaagtaagaaatactatgtagactccgtgaagggccgcttcaccatctccagagacaattccaagaacacgctgtatctgcaaatgaacagcctgagagccgaggacacggctgtgtattactgtgcgagacaaatgggctactggcacttcgatctctggggccgtggcaccctggtcactgtctcctca

SEQ ID NO: 8 - protein, 118 aa, Homo sapiens [sequence omitted]

SEQ ID NO: 9 - DNA, 324 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaa

SEQ ID NO: 10 - protein, 108 aa, Homo sapiens [sequence omitted]

SEQ ID NO: 11 - protein, 6 aa, Homo sapiens
AERSKT

SEQ ID NO: 12 - protein, 11 aa, Homo sapiens
RASQGISSVLA

SEQ ID NO: 13 - protein, 7 aa, Homo sapiens
DASSLES

SEQ ID NO: 14 - protein, 9 aa, Homo sapiens
QQSNSYPLT

SEQ ID NO: 15 - protein, 5 aa, Homo sapiens
SYAIS

SEQ ID NO: 16 - protein, 17 aa, Homo sapiens
GIIPLFDTTKYAQKFQG

SEQ ID NO: 17 - protein, 16 aa, Homo sapiens
DRDILTDYYPMGGMDV

SEQ ID NO: 18 - protein, 8 aa, Homo sapiens [sequence omitted]

SEQ ID NO: 19 - protein, 125 aa, Homo sapiens
QVQLVQSGAEVKKPGSSVKVSCKASGGTFSSYAISWVRQAPGQGLEWMGGIIPLFDTTKYAQKFQGRVTITADESTSTAYMELSSLRSEDTAVYYCARDRDILTDYYPMGGMDVWGQGTTVTVSS

SEQ ID NO: 20 - protein, 108 aa, Homo sapiens
AIQLTQSPSSLSASVGDRVTITCRASQGISSVLAWYQQKPGKAPKLLIYDASSLESGVPSRFSGSGSGTDFTLTISSLQPEDFATYYCQQSNSYPLTFGGGTKVEIKR